(19) United States
(12) Patent Application Publication
(10) Pub. No.: US 2012/0089093 A1
(43) Pub. Date: Apr. 12, 2012

(54) SEAL ARRANGEMENT FOR MINIMALLY INVASIVE DIAGNOSTIC OR SURGICAL INSTRUMENTS

(75) Inventor: Robert M. Trusty, Cincinnati, OH (US)

(73) Assignee: Ethicon Endo-Surgery, Inc., Cincinnati, OH (US)

(21) Appl. No.: 12/900,132

(22) Filed: Oct. 7, 2010

(51) Int. Cl. A61B 17/34

(52) U.S. Cl. 604/164.04

(57) ABSTRACT

A sealing device is disclosed which is intended for use with an access device, such as a trocar, canula or the like, that passes from the outside to the inside of a patient, through an incision in the patient during a minimally invasive diagnostic or surgical procedure to secure one or more tethers, tubes, or the like to the exterior surface of the access device. The sealing device secures one or more tethers in position between the access device and an incision by creating a smooth profile for the access device and tethers and substantially seals against insufflation leakage.

### BACKGROUND

i. Field of the Invention

The present application relates to methods and devices for use in minimally invasive surgical procedures and, more particularly, to a sealing device and method for its use for securing one or more tethers or the like to a portion of the exterior surface of an instrument passing through a port into a patient's body.

ii. Description of the Related Art

In minimally invasive procedures, instruments may be introduced into a site of interest within a patient's body in several ways. Endoscopic minimally invasive surgical and diagnostic medical procedures are used to evaluate and treat internal organs by inserting a small tube into the body. The endoscope, which may have a rigid or a flexible tube, can be used to observe surface conditions of internal organs, including abnormal or diseased tissue such as lesions and other surface conditions and capture images for visual inspection and photography. The endoscope may be adapted and configured with working channels for introducing medical instruments to the treatment region for taking biopsies, retrieving foreign objects, and/or performing surgical procedures.

One form of endoscopic surgery is natural orifice transluminal endoscopic surgery (NOTES™), wherein surgical instruments and viewing devices are introduced into a patient's body with a flexible endoscope through, for example, the mouth, nose, anus, and/or vagina. In another form of minimally invasive procedure, a trocar may be introduced into a patient's body through a relatively small—keyhole—incision (usually 0.5-1.5 cm).

In laparoscopic surgery, a surgeon may place one or more small ports into a patient's abdomen to gain access into the abdominal cavity of the patient. A surgeon may use, for example, a port for insufflating the abdominal cavity to create space, a port for introducing a laparoscope for viewing, and a number of other ports for introducing surgical instruments for operating on tissue.

In Single Site Laparoscopic (SSL) surgery, also known as Single Incision Laparoscopic Surgery (SILS) and Single Port Surgery (SPL) among others, a surgeon may create a single incision in the abdomen and place a single device in that incision that contains multiple ports for introducing surgical instruments for operating on tissue (e.g., a wound retractor).

One class of such devices is magnetically-based (MACS). MACS devices typically include an internal end-effector that provides therapy to the patient (e.g. electro-cautery) or information to the surgeon (e.g. a video camera) and an external magnet used by the surgeon to control the internal device.

Some of the devices delivered through the port may be electronic in nature and require power or electronic data to be delivered to make the devices operable. For example, devices such as a motorized stapler, RF ablation device, harmonic scalpel, or bi-polar forceps may make use of an electronic connector. Still other devices may require delivery of a fluid, either or both of a gas or a liquid, delivered from a source outside of the patient, or it may be necessary to withdraw fluids from the operative site within the patient during the procedure. One means for accomplishing the transfer of electronic data, images or signals, or the transfer of fluids is for the delivered device to include, as appropriate, an electronic tether, wires, or tubing for fluid passage.

If the tether, wire, or tube is placed along-side of an instrument defining an access port, between the port and the patient's abdominal tissue, a gap between the tissue and the port may be created. This gap will allow insufflation to leak from the patient's abdomen, reducing visibility and surgical access. This issue can be worsened when multiple tethers are in place, as the tethers may tent the tissue away from the trocar, especially if they are directly adjacent to each other.

The foregoing discussion is intended only to illustrate various aspects of the related art in the field of the invention at the time, and should not be taken as a disavowal of claim scope.

### SUMMARY

To minimize the size and number of such incisions, it is desirable to deliver devices into the patient's body via the access ports in a way that allows the port to be available for other uses once the device is delivered.

A sealing device for use with an access device and one or more tethers, wires, tubing or the like that pass, in use, along the side of an access device defining an access port of a patient during a minimally invasive diagnostic or surgical procedure is described herein. The sealing device fills and seals any gaps that may be created by tethers running along the side of the access device. The sealing device is molded or fitted onto at least a portion of the circumference or outer perimeter of an access device over one or more tethers to sandwich the tethers, wires, tubing and the like, between the access device and sealing device, or between the sealing device and the tissue defining the walls of the incision.

One embodiment of the sealing device includes a flexible panel having a first surface for conforming to at least a portion of an exterior surface of the access device and a second surface for contact, in use, with an incision in a patient, the panel being structured to secure at least one tether between the panel and one of the exterior surface of the instrument or the incision.

The sealing device may have at least one slot therein for receiving a portion of a tether. The one or more slots may be positioned on the first surface of the flexible panel, on the second surface of the flexible panel, or there may be at least one slot positioned on each of the first and second surfaces of the flexible panel.

The slots may have one or more engaging members, such as ribs or another form of protrusion from the surface of the slot, for releasably securing the tether within the slot. In another embodiment, there may be no engaging members. The one or more tethers may be press fit into a complementarily-sized slot. In one embodiment, each slot may be formed from an elongate groove cut along the length of the flexible panel. In another embodiment, or in the same embodiment, the slot may be formed from an elongate groove positioned generally diagonally along the flexible panel to allow the tether to partially or fully spiral around the access device as the tether passes through the access port from the inside to the outside of the patient. In another embodiment, the flexible panel may be overmolded onto the one or more tethers and completely encompass the one or more tethers all the way around.

The flexible panel, which may be made of a biocompatible polymer or rubber, may be molded onto at least a portion of the exterior surface of the access device. Alternatively, the sealing device may be preformed to conform to the shape of at least a section of the circumference or outer perimeter of at least a portion of the exterior surface of the access device. In another embodiment, the sealing device may be molded or fitted onto the access device to completely encase the circumference or outer perimeter of the portion of the exterior surface of the access device that may pass through the access port. The flexible panel may be preformed, in cross-section, into an arcuate shape contoured for a grasping fit about at least a portion of the access device. In any of the foregoing embodiments, the flexible panel may have been overmolded onto the one or more tethers to completely encompass the one or more tethers all the way around prior to being molded onto the access device or prior to or during the formation of the sealing device. In one embodiment, the access device may be a trocar and the flexible panel may be preformed to an arcuate shape, in cross-section, to conform to the shape of at least a portion of the exterior surface of the trocar.

In another embodiment, the access device may be a SSL port and the flexible panel may be preformed into an arcuate shape that conforms longitudinally to the flexible tissue wound retractor portion of the port.

Also provided is a kit that includes an access device, such as, for example, a trocar or canula, and a sealing device for securing one or more tethers to the access device. The kit may have differently sized sealing devices to conform to differently sized access devices and to accommodate one, two or more tethers. Plugs for sealing unused slots in the sealing device may also be provided.

### FIGURES

Various features of the embodiments described herein are set forth with particularity in the appended claims. The various embodiments, however, both as to organization and methods of operation, together with advantages thereof, may be understood in accordance with the following description taken in conjunction with the accompanying drawings as follows.

FIG. 1 illustrates an embodiment of a sealing device having an external slot for receiving a tube, tether, wire or the like, and overmolded onto an internal tube, tether, wire or the like that runs along a portion of the length of a trocar.

FIG. 2 illustrates the trocar and sealing device of FIG. 1 passing through an abdominal wall.

FIG. 3 illustrates an alternative embodiment of the sealing device of FIG. 1 showing two external and two internal slots.

FIG. 4 illustrates an alternative embodiment of the sealing device of FIG. 1 showing two tethers press fit between the trocar and the sealing device.

FIGS. 5 A-C illustrate front, side section, and top plan views of an embodiment of a flexible sealing device having external and internal slots with ribbed edges for releasably capturing a tube, tether, wire or the like.

FIG. 6 illustrates an embodiment of the sealing device of FIG. 1 with an optional plug inserted into an unused slot.

FIG. 7 illustrates an embodiment of the sealing device applied to a single site laparoscopic port.

Corresponding reference characters indicate corresponding parts throughout the several views. The exemplifications set out herein illustrate various embodiments of the invention, in one form, and such exemplifications are not to be construed as limiting the scope of the invention in any manner.

### DESCRIPTION

Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” or “an embodiment”, or the like, means that a particular feature, structure, or characteristic described in connection with the embodiment is included in at least one embodiment. Thus, appearances of the phrases “in various embodiments,” “in some embodiments,” “in one embodiment,” or “in an embodiment”, or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

When a video camera is delivered either via a NOTES pathway or transcutaneously through a trocar, a wound retractor, an access port or an incision, the ability to remove and clean the lens without withdrawing the camera from the patient becomes difficult. It is possible that at some point in a procedure, the camera lens will become smudged with bodily fluids or material, and a means for clearing the lens to restore unimpeded vision is desirable. A device for cleaning a camera lens may include a nozzle built into an attachable body that is mounted to the camera. The nozzle is placed near the lens and produces a water jet and suction action that effectively cleans the camera lens. The water and suction are delivered via a flexible, hollow-tubed tether, separate from the electronic tether associated with the camera. The flexible tubing and the electronic tether must be connected to sources of cleaning fluid and power outside of the patient's body.

To avoid having to use the space within a trocar, canula, wound retractor, laparoscope or other similar access device, or to take the place of a trocar or such other access device, a sealing device 20 is disclosed herein. The sealing device 20 is intended for use with such access devices for surgical or diagnostic use that pass from the outside to the inside of a patient, through an incision cut into the patient's tissue, such as the abdominal or pelvic wall, to secure one or more tethers, tubes, wires, or the like to the exterior surface of the access device.

In one embodiment of a method of deployment, a working instrument, such as a camera, may be inserted through an incision in a patient's tissue intended for the access device, with one or more tethers trailing behind. An access device, for example, a trocar or a wound retractor, may be re-inserted with the tethers located between the exterior surface of the trocar or wound retractor and the tissue defining the walls of the incision. In several experiments using several different types of trocar, including standard rigid laparoscopic trocars and various single site laparoscopic ports that utilize wound protectors, insufflation leakage between the outer wall of the trocar or wound protector and the tether was observed. The sealing device 20 described herein provides a seal that may be overmolded onto the tethers to smooth out the tether profile and seal against insufflation leakage by substantially sealing the space between the access device and the patient's abdominal tissue.

A smooth profile as used herein means a profile that in cross section has continuous generally arcuate lines, with no irregular contours therein that would create or allow anything but minimal air gaps. Sealing or seal, as used herein, means substantially sealing or a substantial seal such that an absolute seal against all air flow is not necessary or required, recognizing that some relatively small space creating minimal air gaps and some flexibility in the opening to allow the trocar, sealing device and tethers to be moved proximally or distally or at an angle within the incision and to be removed entirely when appropriate, can be tolerated. For example, in an operating room setting, insufflation is obtained and maintained using standard surgical insufflators connected via a flexible tube to an insufflation port on a trocar or SSL port. The insufflator both monitors and maintains insufflation pressure in a closed-loop-control fashion. Typically, the insufflator is set to automatically maintain 15 mmHg pressure in the patient's abdomen. Leak rates are tolerated as long as the insufflator can keep up and maintain good visualization and access.

Access port as used herein refers to and shall include, without limitation, an opening defined in an instrument creating a passage between the outside surface and an internal site of the patient, such as an internal cavity, lumen (such as a vein or artery, bowel, esophagus, etc.), or organ of a patient, used during a surgical or diagnostic procedure. The access port may pass through an incision or through a natural orifice sized for minimally invasive surgery or diagnostic procedures.

An incision, as used herein, is defined by the walls of the tissue surrounding it and creates a passage between the outside surface and an internal site of the patient, such as an internal cavity, lumen (such as a vein or artery, bowel, esophagus, etc.), or organ of a patient used during a surgical or diagnostic procedure. An incision sized for minimally invasive surgery or diagnostic procedures may be generally a keyhole incision of about 0.5 to 2.5 cm, but those skilled in the art will recognize that the size of the incision may be smaller or larger, as appropriate for the size of the patient, the site of and kind of procedure, and the size of the instruments to be inserted through the access port. The incision may be lined with a wound protector. Instrument and tethers pass through the incision, along either side of the wound protector.

Tether, as used herein, refers to and shall include, without limitation, any tether, tubing, wires or the like passing between an internal site of the patient and the outside surface.

As used herein, the term “patient” refers to any human or animal on which a procedure may be performed. As used herein, the term “internal site” of a patient means a lumen, body cavity or other location in a patient's body including, without limitation, sites accessible through natural orifices, access ports, or through incisions.

The sealing device 20 secures sections of one or more tethers in position between the access device and the incision by means of one or more of the following methods: by capturing the tether within a slot formed in the sealing device, by creating a pressure fit between the exterior surface of the access device and the facing interior surface of the sealing device 20, or by creating a pressure fit between the exterior surface of the sealing device and the incision.

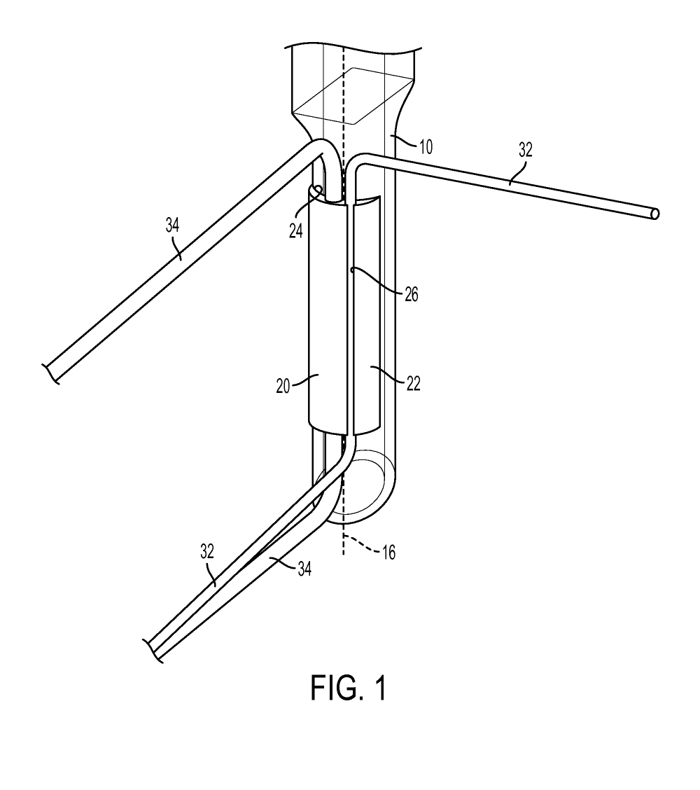

FIG. 1 is a view of an embodiment of the sealing device 20 attached to an access device. The access device may be a trocar 10 having a longitudinal axis 16, and exterior surface 14 and an internal channel or passage way. In this embodiment, the sealing device 20 includes an external slot or groove 26 for receiving and securing therein a section of a tether 32. Another section of tether 34 may be captured between the exterior surface 14 of the trocar 10 and the interior surface 24 of the sealing device 20. The sealing device 20 may be overmolded onto a section of the circumference or outer perimeter of the exterior surface 14 of the trocar 10. Alternatively, the sealing device 20 may be preformed to conform to the shape of the exterior of the access device to create a grasping fit. In either embodiment, where the sealing device 20 is overmolded onto the access device or preformed to a desired shape, the sealing device 20 may have zero, one, two or more slots 26/28 on one or both of the interior or exterior surfaces 24/22 of the sealing device 20.

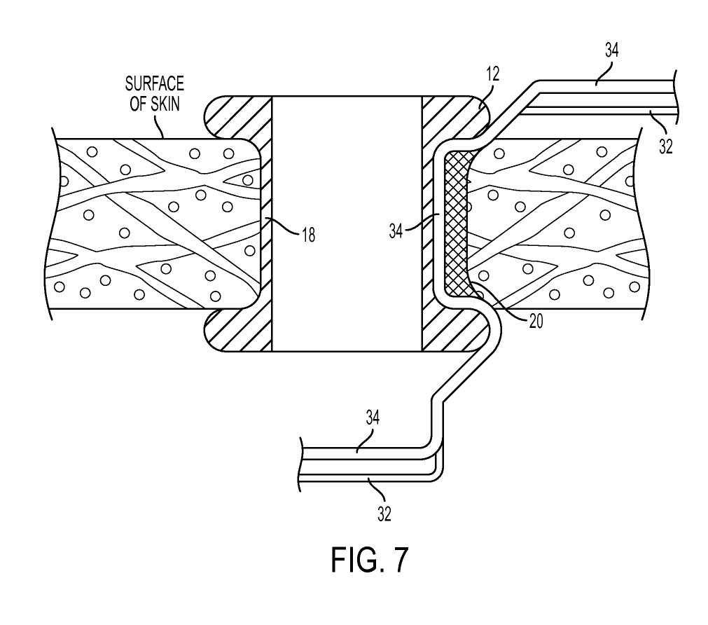

A sealing device 20 is shown in FIG. 7 with an alternative access device, wound retractor 12. Wound retractor 12 is the portion of an access device used in SSL surgery that is inserted in the incision. A cap (not shown) having several openings for working instruments would be connected to the wound retractor 12. In the arrangement shown in FIG. 7, the sealing device 20 is overmolded onto one or more tethers 32/34 and seals the area around the tethers between the groove 18 of wound retractor 12 and the tissue of the incision. The ends of the tether 32 extend distally and proximally beyond each rim of the wound retractor 12.

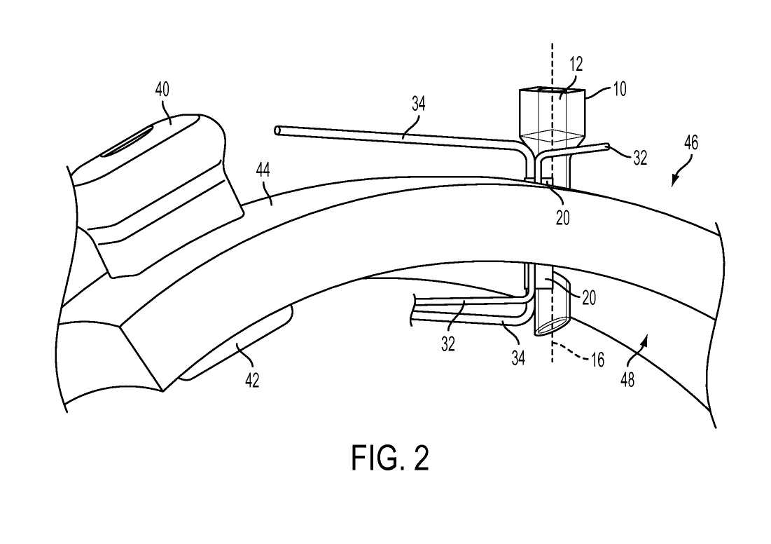

FIG. 2 illustrates the sealing device 20 in use. The sealing device 20 conforms to a section of the exterior shape of the portion of the trocar 10 that passes through the incision. The sealing device 20 holds tether 34 between the interior surface 24 of the sealing device 20 and the exterior surface 14 of the trocar 10. Sealing device 20 holds the tether 32 between the exterior surface 22 of the sealing device 20, or, as shown, within the slot 26 in the exterior surface 22 of the sealing device 20, and the incision through, for example, the abdominal wall 44. The trocar 10 will press into the sealing device 20 and retain it in place through friction and the exterior surface the sealing device 20 will slip against the tissue as the trocar 10 is inserted or withdrawn. The tethers 32, 34 extend from the magnet sled 42 inside the patient, for example, in the abdominal cavity 48 through the incision in the abdominal wall 44 to the outside 46 of the patient's body. The internal magnet sled 42 may house or be attached to a camera or another working instrument. Outside of the body, an external magnet 40 may be used to manipulate the internal magnet sled 42, and thereby, the working instrument attached to or housed therein. One tether 34, for example, may connect to a water source or a source of another type of biocompatible lens cleaner. The other tether 32, for example, may connect to a power source or may connect to a receiver to receive video images for display on a video screen in the operating room or to a remote location, or both.

The incision is a relatively small keyhole incision, typically about 0.5 to 2.5 cm, and more preferably 0.5 to 1.5 cm, and most preferably 1.0 to 1.5 cm, sized to allow a trocar or a like sized access device to pass through with little, and preferably no more excess space than necessary to allow passage of a desired number of tethers 32, 34. The sealing device 20, as stated above, creates a smooth profile by pressing one or both of the tethers 32, 34 against the exterior of the trocar 10, or between the sealing device 20 and the walls defining the incision to minimize, or preferably prevent, the formation of air pockets or air passages around the curvature of the tethers 32, 34. The incision exerts sufficient force against the sealing device 20 that the tethers 32, 34 are in effect sandwiched between the incision and the trocar 10, on the interior side 24, exterior side 22, or both sides 22, 24 of the sealing device 20. The pressure or force exerted against the tethers 32, 34, however, can not be so great as to crimp the tether 32, 34 to interfere with the flow of fluid, power or video signals, passing through the tether.

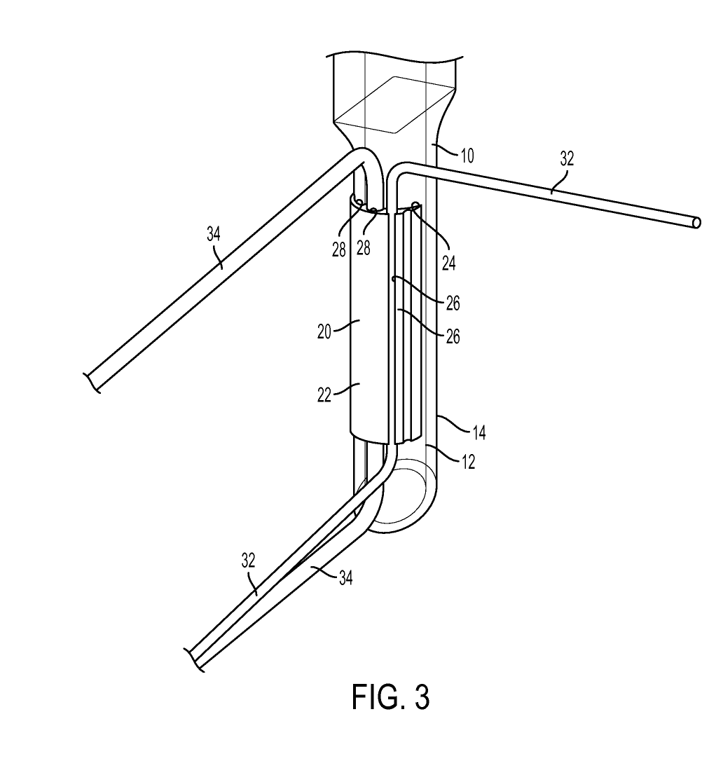

In another embodiment, as shown in FIG. 3, the sealing device 20 may have two external slots 26 and two internal slots 28 for receiving and securing therein up to four tethers 32, 34.

In one embodiment, each slot 26, 28 may be formed from an elongate groove cut along the length of the flexible panel, generally parallel to the axis 16 of the trocar 10. In another embodiment, or in the same embodiment, one or more of the slots 26, 28 may be formed from an elongate groove positioned generally diagonally along the flexible panel to allow one or more of the tethers 32, 34 to partially or fully spiral around the trocar 10 as the tether 32, 34 passes through the incision from the inside to the outside of the patient.

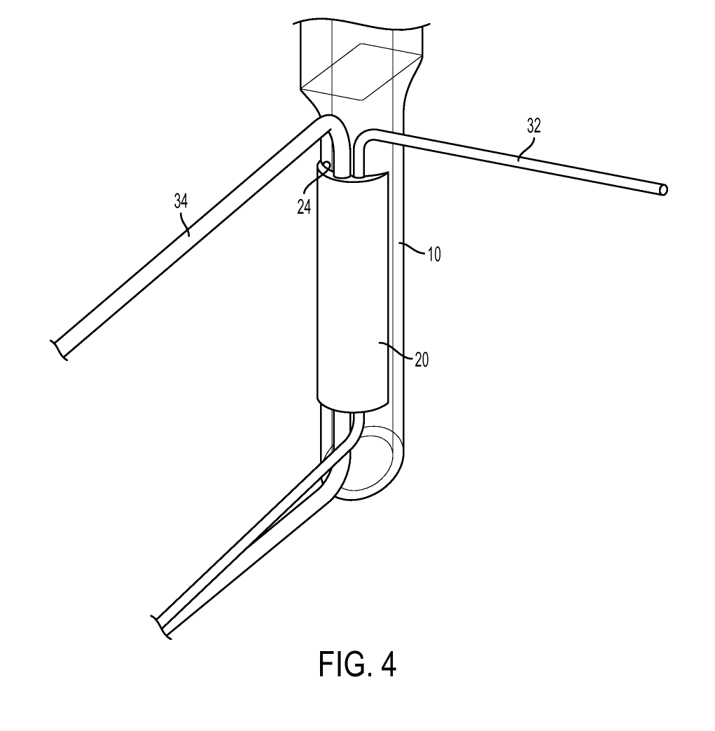

In the embodiment shown in FIG. 4, there are no external slots 26. One, two or more tethers 32, 34 may be secured against trocar 10 by overmolding the sealing device 20 over the one or a plurality of tethers 32, 34 and a sufficient portion of the exterior 14 of the trocar 10 to capture the tethers 32, 34 and create a smooth profile against trocar 10 for a substantially gap-free passage through the incision in the abdominal wall 44. In this embodiment, there are no slots formed in the sealing device 20. Alternatively, there may be one, two or more slots 28 on the interior side 24 of sealing device 20 for receiving the tethers 32, 34.

Those skilled in the art will appreciate that one, two or more tethers may be captured by sealing device 20 against at least a portion of the exterior surface 14 of the trocar 10, or that zero, one, two or more slots may be formed on the interior side 22, the exterior side 24, or on both sides 22, 24 of the sealing device 20 to secure a desired number of tethers 32, 34, as needed in any given procedure.

The sealing device 20 is preferably made of a biocompatible, flexible polymer material such as polypropylene, silicon, thermoplastic elastomer (TPE), or TEFLON®. The material may be sufficiently malleable to be pressed into gaps surrounding the curvature of tethers 32, 34 and the surface texture and contours of the trocar 10 to substantially seal against gaps. Those skilled in the art will also recognize that a sealing device 20 may also be rigid in nature and be made of biocompatible rigid materials, such as stainless steel or rigid polymers, for example polycarbonate, glass-filled polycarbonate or nylon.

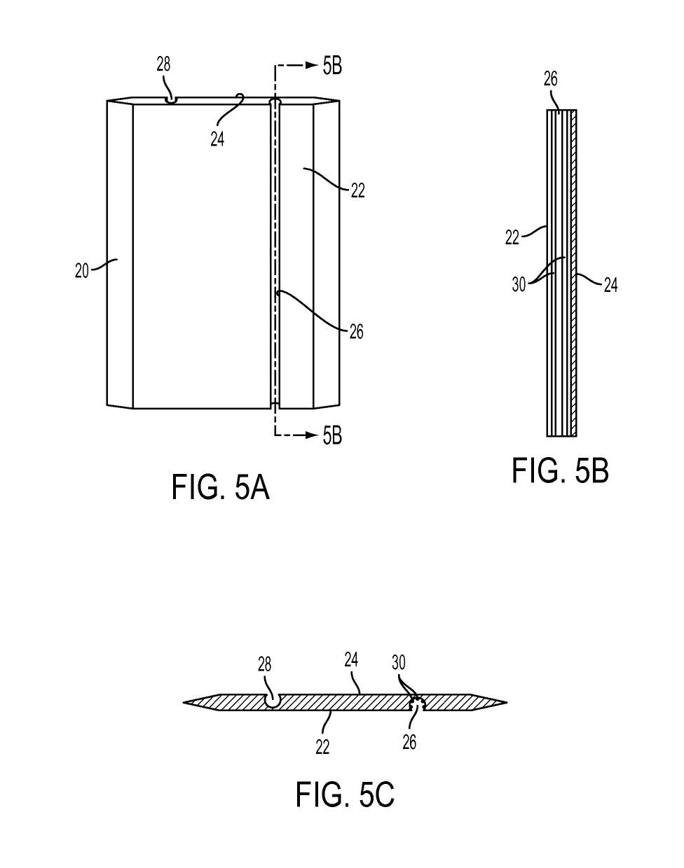

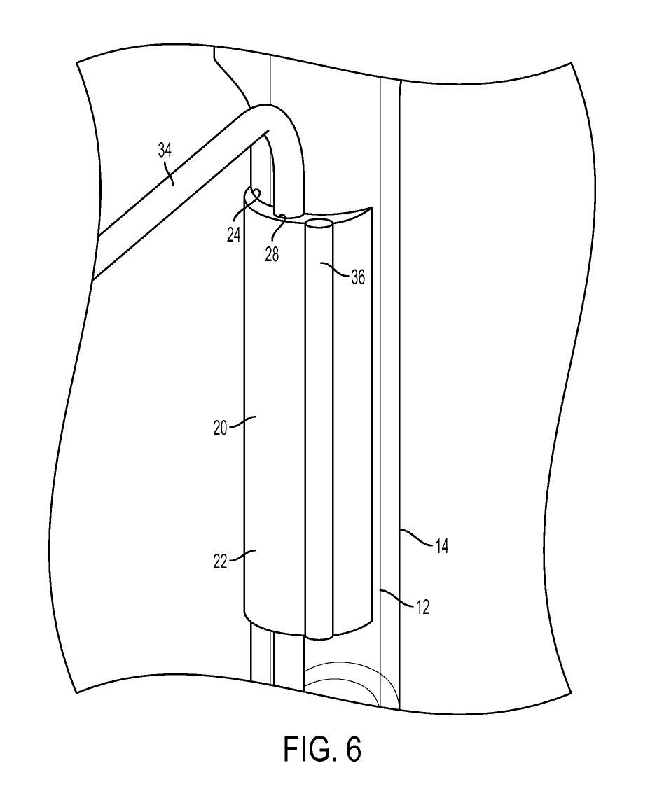

As shown in FIG. 5, sealing device 20 may be a sealing member having a relatively flat, but flexible, moldable, panel having front 22 and back 24 surfaces. The edges along the longitudinal axis of the panel may be tapered to further create a smooth profile along the length of the access device when attached. At least one slot 26, 28 may be cut into each of one or both surfaces 22, 24 of the panel for receiving tethers 32, 34. Alternatively, a plurality of slots 26, 28 may be cut into only one side or into both sides 22, 24 of the sealing device panel. Slot plugs 36 that conform to and fill at least a part of the space created by the slots 26, 28 may be provided when fewer than the available slots are needed to seal against air flow in an unused slot 26, 28. See FIG. 6.

The slots 26, 28 may have one or more engaging members 30, such as ribs or another form of protrusion, from the surface of the slot 26, 28, for releasably securing the tether 32, 34 within their respective slots 26, 28. In another embodiment, there may be no engaging members 30. The one or more tethers 32, 34 may be press fit into a complementarily-sized slot.

The sealing device may alternatively be preformed in a shape that conforms to the exterior shape of a trocar. Different overmolds can be designed to work with a multitude of sizes and designs of trocars and ports. Trocars and like access devices are made in different sizes and may be circular, oval, or irregular in cross-section, but generally have smooth exterior surfaces to avoid trauma to tissue. Sealing devices 20 may be provided separately or sold in kit assemblies in a variety of lengths, circumferences, radiused arcs, and preformed shapes in cross-section, to conform to and provide a grasping fit to form a seal against at least a section of the circumference or outer perimeter of at least a portion of the exterior surface of the access devices in the kit.

In one embodiment, a sealing instrument is provided that includes an access device, such as trocar 10, with a flexible sealing member structured for releasably securing one or more tethers 32, 34 of a working instrument, such as a camera, to the access device to together define a smooth profile for sealing the incision. The flexible sealing member may have all or any combination of the features of the sealing device 20 described herein, and like sealing device 20, may be a panel that can be overmolded onto the access device or may be preformed to conform to at least a portion of the exterior shape of the access device.

The sealing device 20 may be molded into a single unit when overmolding onto a tether 10 or extruded and cut to length when a slotted version is produced. However, those familiar with the art will recognize that the several embodiments of the sealing devices 20 may also be produced by machining, laser cutting, grinding, hot-pressing, or other equally suitable means.

In one embodiment of a method of deployment, a working instrument, such as a camera, may be inserted through an incision, with one or more tethers 32, 34 trailing behind, with one end and a suitable length of the tether on the outside of the patient's body. A trocar 10, for example, may be re-inserted with the tethers 32, 34 sandwiched between the exterior surface 14 of the trocar 10 and the incision by the aid of the sealing device 20 described herein.

Prior to re-insertion of the trocar 10, the appropriately sized sealing device 20 is attached to the trocar 10 to secure the tethers 32, 34 against the trocar 10, thereby smoothing out the profile of the one or more tethers 32, 34 against the trocar 10. In embodiments of sealing device 20 having one or more slots 26, 28, the tether or tethers 32, 24 are inserted into the one or more slots 24, 26 and the sealing device 20 is attached to the trocar 10. In embodiments of sealing device 20 without slots 28 on the interior side 24, the sealing device 20 may be overmolded or press fit onto the exterior 14 of the trocar 10 over the one or more tethers 32, 34. If there are one or more slots 26 on the exterior 22 of the sealing device 20, the one or more tethers 32, 34 are inserted into an appropriate slot 26. After attachment of the sealing device 20 with the tethers 32, 34 secured, the trocar 10 is inserted into the incision. If the sealing device 20 has no slots 26 on the exterior 22 of the sealing device, and there are one or more tethers 32, 34 to be positioned between the exterior 22 of the sealing device 20 and the incision, the trocar with sealing device 20 attached are carefully inserted through the incision along side the exterior tethers to press the tethers 32, 34 between the exterior 22 of the sealing device 20 and the incision and thereby create a smooth profile within the incision and substantially seal the incision against insufflation leakage. The sealing device 20 creates a smooth profile around the trocar 10 and within the incision that will minimize or eliminate air gaps between the curvature of each tether 32, 34 and the surrounding trocar 10 and incision.

Preferably, the various embodiments of the devices described herein will be processed before surgery. First, a new or used instrument is obtained and if necessary cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility. Other sterilization techniques can be done by any number of ways known to those skilled in the art including beta or gamma radiation, ethylene oxide, and/or steam.

Although the various embodiments of the devices have been described herein in connection with certain disclosed embodiments, many modifications and variations to those embodiments may be implemented. For example, different types of working instruments with different types of tethers may be employed and different types of access devices may be employed. Also, where materials are disclosed for certain components, other materials may be used. The foregoing description and following claims are intended to cover all such modification and variations.

What is claimed is:

1. A sealing device for use with an access device and one or more tethers passed, in use, through an incision in a patient during a minimally invasive diagnostic or surgical procedure, the sealing device comprising:
a flexible panel having a first surface for conforming to at least a portion of an exterior surface of the instrument and a second surface for contact, in use, with an incision in a patient, the panel being structured to secure at least one tether between the panel and one of the exterior surface of the instrument or the incision.

2. The sealing device recited in claim 1 wherein there is at least one tether secured between the first surface of the panel and the exterior surface of the instrument and at least one tether secured between the second surface and the incision.

3. The sealing device recited in claim 1 wherein the at least one tether is positioned between the first surface of the panel and the exterior surface of the instrument and the flexible panel is overmolded onto the at least one tether and the instrument.

4. The sealing device recited in claim 3 further comprising at least one slot formed on at least one of the first or the second surfaces of the flexible panel for receiving a section of a tether.

5. The sealing device recited in claim 1 further comprising at least one slot in the panel for receiving a section of a tether.

6. The sealing device recited in claim 5 wherein the at least one slot is positioned on one of the first or the second surfaces of the flexible panel.

7. The sealing device recited in claim 5 wherein there is at least one slot positioned on each of the first and second surfaces of the flexible panel.

8. The sealing device recited in claim 5 wherein the at least one slot has engaging members along at least a portion thereof for releasably securing the tether within the slot.

9. The sealing device recited in claim 8 further comprising a plug for each slot for sealing the slot when the slot is not occupied with a tether.

10. The sealing device recited in claim 5 wherein the slot is an elongate slot along the length of the flexible panel.

11. The sealing device recited in claim 5 wherein the slot is an elongate slot positioned generally diagonally along the flexible panel.

12. The sealing device recited in claim 1 wherein the flexible panel is preformed to conform to the shape of a portion of the exterior surface of the instrument.

13. The sealing device recited in claim 1 wherein the instrument is a trocar and the flexible panel is preformed to an arcuate shape, in cross-section, to conform to the shape of at least a portion of the exterior surface of the trocar.

14. The sealing device recited in claim 1 wherein the flexible panel is made of a biocompatible polymer.

15. A sealing instrument for use in an incision in a patient during a minimally invasive procedure comprising:
an access device having an interior channel and an exterior surface; and
a flexible sealing device structured for releasably securing one or more tethers of a working instrument between the exterior surface of the access device and, in use, the incision, to define a smooth profile for the access device and the one or more tethers for sealing the incision.

16. The sealing instrument recited in claim 15 wherein the sealing device comprises a flexible panel having a first surface for conforming to at least a portion of an exterior surface of the working instrument and a second surface for contact, in use, with the incision in a patient.

17. The sealing instrument recited in claim 16 wherein the at least one tether is positioned between the first surface of the panel and the exterior surface of the instrument and the flexible panel is overmolded onto the at least one tether and the instrument.

18. The sealing instrument recited in claim 16 further comprising at least one slot formed on one or both of the first and the second surfaces of the flexible panel for receiving a section of the tether.

19. The sealing instrument recited in claim 18 wherein the at least one slot has engaging members along at least a portion thereof for releasably securing the tether within the slot.

20. A method for securing a tether-like member to an access device for passage through an incision in a patient, the method comprising:
providing a sealing device having a first surface for conforming to at least a portion of an exterior surface of the access device and a second surface for contact, in use, with an incision in a patient;
running at least one tether along one or both of the exterior surface of the access device and the exterior surface of the sealing device;
attaching the sealing device over at least a portion of the exterior surface of the access device to define a smooth profile; and,
inserting the access device having the one or more tethers and sealing device attached thereto through the keyhole incision to seal the incision and secure the one or more tethers between the keyhole incision and the access device.
